(19) United States
(12) Patent Application Publication
(10) Pub. No.: US 2024/0335203 A1
(43) Pub. Date: Oct. 10, 2024

(54) METHODS FOR DRILLING-GUIDEWIRE TARGETING OF CANNULATED DEVICES

(71) Applicant: ExsoMed Corporation, Aliso Viejo, CA (US)

(72) Inventors: Larry Ehmke, Portland, OR (US); Michael Zwolinski, Willoughby, OH (US)

(21) Appl. No.: 18/629,482

(22) Filed: Apr. 8, 2024

Related U.S. Application Data
(60) Provisional application No. 63/458,334, filed on Apr. 10, 2023.

(51) Int. Cl. A61B 17/16; A61B 17/17; A61B 17/56; A61M 25/09

(52) U.S. Cl. CPC A61B 17/1631; A61B 17/1615; A61B 17/1728; A61B 2017/564; A61M 2025/09183

(57) ABSTRACT

Provided herein are methods for inserting a bone fastener into a bone and securing an implant using a drilling guidewire. The method may include positioning a first bone plate having a first hole on a first side of a bone and drilling a channel through the bone from the first side of the bone to a second side of the bone using a drilling guidewire. The drilling guidewire may include a first portion having a drill unit and a second portion, the second portion extending from the second end of the first portion. Additionally, the diameter of the first portion may be greater than a diameter of the second portion. The method may further include sliding a bone fastener over the second portion of the drilling guidewire and driving the bone fastener through the first hole of the first bone plate and the channel in the bone.

### PRIORITY CLAIM

This application claims priority to U.S. Provisional Patent Application No. 63/458,334 filed on Apr. 10, 2023, the entire contents of which are hereby incorporated by reference and relied upon.

### BACKGROUND

Currently, securing implants such as bone plates or intramedullary screws to the bone requires multiple steps and different tools. For example, to secure a bone plate to a bone, surgeons typically first solid drill through the plate hole and out the far cortex, remove the drill, and insert a screw into the drilled hole.

With the above-described method, it can be challenging to find the near cortical hole to insert the screw into and to accurately insert the screw through the bone and out the far cortical hole. This can be even more difficult when there is a second bone plate on the far side of the bone through which the screw needs to traverse.

### SUMMARY

The present disclosure provides a new and innovative drilling guidewire and methods for quick and accurate bone fastener insertion into an implant, such as a bone plate or intramedullary device, using a dual diameter drilling guidewire. In some examples, a method of inserting a bone screw into a bone is provided. The method may include positioning a bone plate on a first side of the bone and drilling a channel from a first side of the bone to a second side of the bone through a hole in the bone plate using a drilling guidewire. The drilling guidewire may include a first portion and a second portion, where the first portion has a greater diameter than the second portion. The first portion may include a drill unit. The method may further include moving the drilling guidewire through channel in the bone until only the second portion of the drilling guidewire is disposed in the bone. A bone fastener may slide over the second portion of the guidewire and be driven through the hole in the bone plate into the channel in the bone.

Additional features and advantages of the disclosed methods are described in, and will be apparent from, the following Detailed Description and the Figures.

### BRIEF DESCRIPTION OF THE FIGURES

FIGS. 1A and 1B are diagrams of example drilling guidewires according to examples of the present disclosure.

FIGS. 2A-2E are diagrams illustrating an example method according to an example of the present disclosure.

FIGS. 3A and 3B are diagrams illustrating an example method according to an example of the present disclosure.

FIGS. 4A and 4B are diagrams illustrating an example method according to an example of the present disclosure.

FIGS. 5A-5C are diagrams illustrating an example method according to an example of the present disclosure.

FIGS. 6A and 6B are diagrams illustrating an example method according to an example of the present disclosure.

FIGS. 7A-7E are diagrams illustrating another example method according to another example of the present disclosure.

FIGS. 8A-8C are diagrams illustrating another example method according to another example of the present disclosure.

### DETAILED DESCRIPTION OF EXAMPLES

The present disclosure is directed to a drilling guidewire and a method for inserting a bone fastener through the bone and securing an implant using the drilling guidewire.

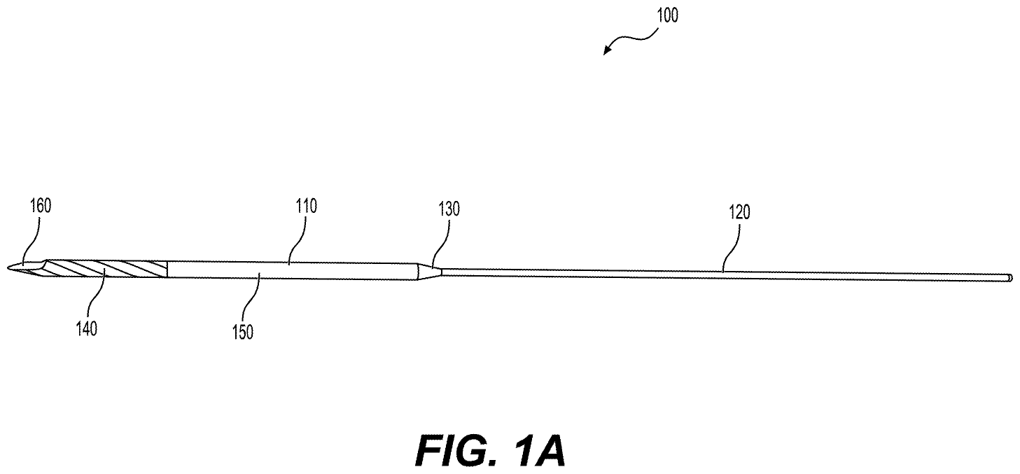

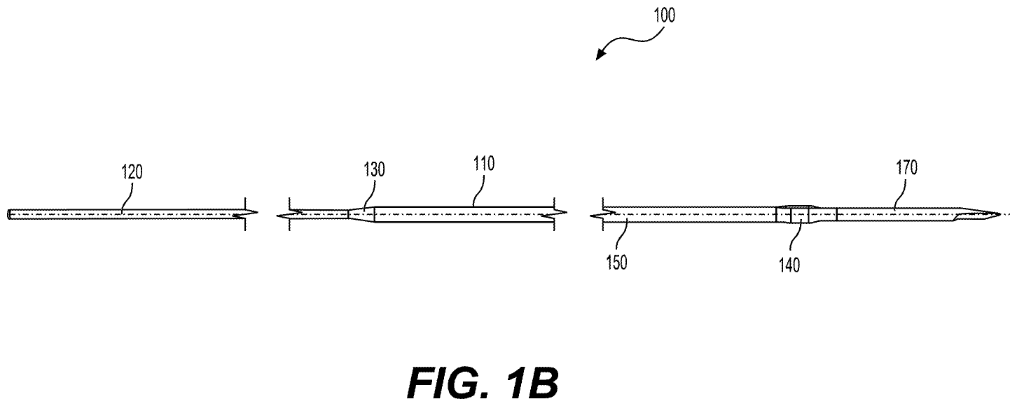

FIG. 1 shows an example drilling guidewire 100 according to an example of the present disclosure. The drilling guidewire 100 may include a first portion 110 and a second portion 120. In some examples, the first portion 110 and/or second portion 120 may have a generally uniform outer diameter. The outer diameter of the first portion 110 may be greater than the uniform outer diameter of the second portion 120.

The first portion 110 may include a first end and a second end. The first end of the first portion 110 may be a free end. The second end of the first portion 110 may be coupled to the second portion 120. In some examples, the second portion 120 may extend from the second end of the first portion 110.

In some examples, the drilling guidewire 100 may include a third portion 130 between the first portion 110 and the second portion 120. The diameter of the third portion 130 may gradually decrease from the diameter of the first portion 110 to the diameter of the second portion 120.

In other examples, the first portion 110 may be connected to the second portion 120 without an intermediate portion therebetween. In this case, the change in diameter from the first portion 110 to the second portion 120 may be abrupt and the portion between the first portion 110 and the second portion 120 may have a stepped configuration.

The first portion 110 may include a drill unit 140 designed to drill a hole or channel through a bone. The drill unit 140 may include one or more flutes (e.g., 1, 2, 3, 4, 5, 6, 7, 8, . . . ). In some examples, the drill unit 140 may include a sharpened tip 160 configured to pierce bone.

In some examples, the drill unit 140 spans the entire first portion 110. In other examples, only an end portion of the first portion 110 (e.g., near the first end of the first portion 110) may include the drill unit 140, and the first portion 110 may include a shaft portion 150 (with a smooth surface) extending from the drill unit 140. The smooth surface of the shaft portion 150 may be configured to easily glide through the channel drilled into the bone by the drill unit 140.

Referring to FIG. 1B, in some examples, the drilling guidewire 100 may include a fourth portion 170 coupled to one end of the drill unit 140 opposite the shaft portion 150. In some examples, the fourth portion 170 may have a smaller diameter than the drill unit 140 and/or the shaft portion 150 and a larger diameter than the second portion 120. The fourth portion is configured to facilitate insertion of the drilling guidewire 100 and to start the trajectory into the bone 10 before drilling by the drill unit 140. In some examples, the diameter of the shaft portion 150 may be slightly smaller than the diameter of the drill unit 140 to limit drag through the bone 10. In some examples, the drill unit 140 may be one or more cutting flutes. In some examples, other exemplary drilling guidewires may be used, such as those disclosed by U.S. patent application Ser. No. 17/929,425 titled ORTHOPEDIC IMPLANTS AND INSTRUMENTS FOR DELIVERING THE SAME, which was filed on Sep. 2, 2022, the disclosure of which is hereby incorporated by reference in its entirety.

The second portion 120 may be configured to receive a bone fastener, such as a cannulated bone fastener. For example, the diameter of the second portion 120 may be substantially the same as or smaller than a cannulation in the bone fastener. Additionally, the first portion 110 may include a diameter greater than the cannulation in the bone fastener, so that the bone fastener will not slide over the first portion 110 of the drilling guidewire 100.

The two diameters may give the drilling guidewire 100 the ability to drill into bone without bending, while still containing a portion with a diameter small enough to receive a cannulated device and guide the device through the channel in the bone. In some examples, the diameter of the first portion 110 may be in the range of about 1.2 mm to about 6.5 mm, for example, about 1.2 mm to about 2.3 mm, about 2.3 mm to about 3.4 mm, about 3.4 mm to about 4.5 mm, about 4.5 mm to about 5.6 mm, about 5.6 mm to about 6.5 mm. In some examples the diameter of the second portion 120 may be in the range of about 0.5 mm to about 4.5 mm, for example, about 0.5 mm to about 1.5 mm, about 1.5 mm to about 2.5 mm, about 2.5 mm to about 3.5 mm, or about 3.5 mm to about 4.5 mm.

In some examples, the ratio between the diameter of the first portion 110 and the diameter of the second portion 120 may be in the range of about 1.4:1 to about 13:1, for example, from about 1.4:1 to about 3.5:1, from about 3.5:1 to about 5.6:1, from about 5.6:1 to about 7.7:1, from about 7.7:1 to about 9.8:1, from about 9.8:1 to about 11.9:1, or from about 11.9:1 to about 13:1. In other examples, the ratio between the diameter of the first portion 110 and the diameter of the second portion 120 may have any other suitable range.

In some examples, a method of inserting a bone fastener using the drilling guidewire 100 disclosed herein according to the present disclosure is provided. The method may allow inserting a bone fastener through a bone more accurately, safely, and in less steps than traditional methods. The steps presented herein may be performed in any suitable order and combination, and they may be modified by or combined with any of the other procedures and features disclosed elsewhere herein.

In some examples, the method may include positioning a bone plate on a first side of the bone. The bone plate may have one or more holes configured to receive a bone fastener. The bone may be a humerus, a radius, an ulna, a clavicle, a tibia, a fibula, a metacarpal, a metatarsal, a phalange, or a femur. It can be appreciated that one skilled in the art would understand additional bones that could be used in the methods disclosed herein. The bone may contain a fracture, a break, or some other imperfection where alignment of the bone is necessary. Any suitable bone plate may be used depending on the type of imperfection and the bone.

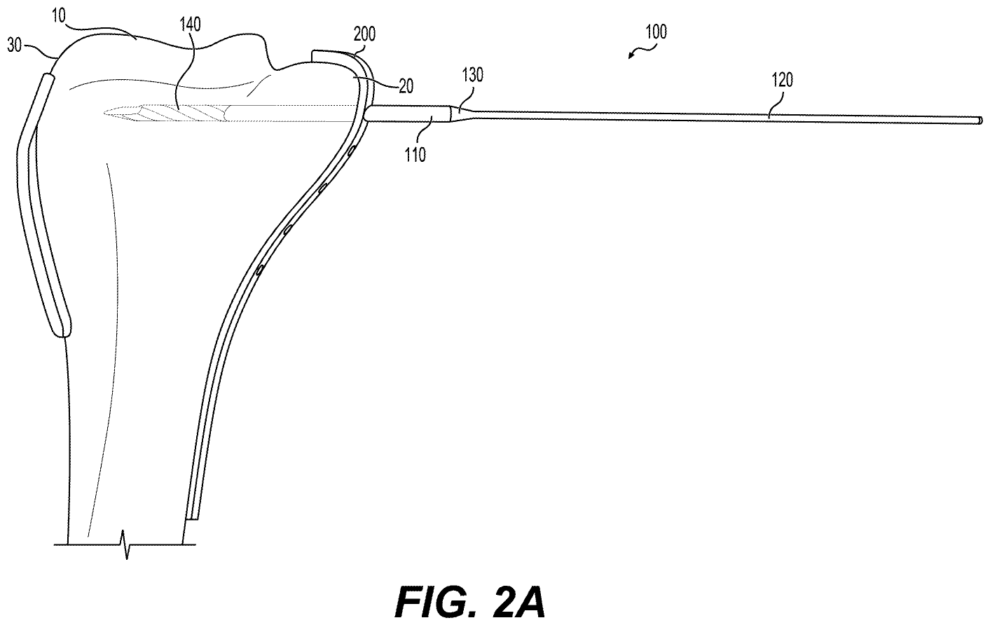

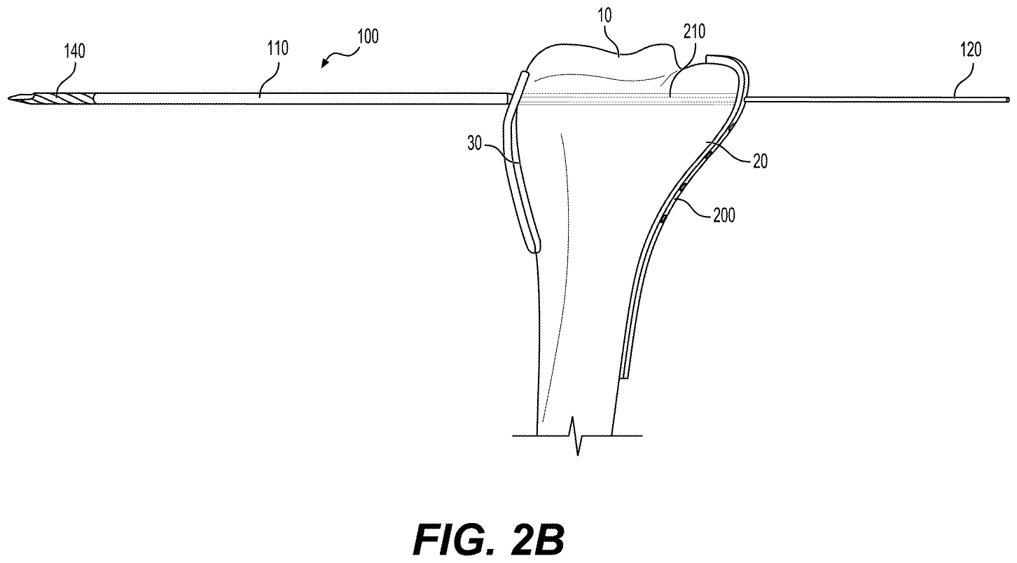

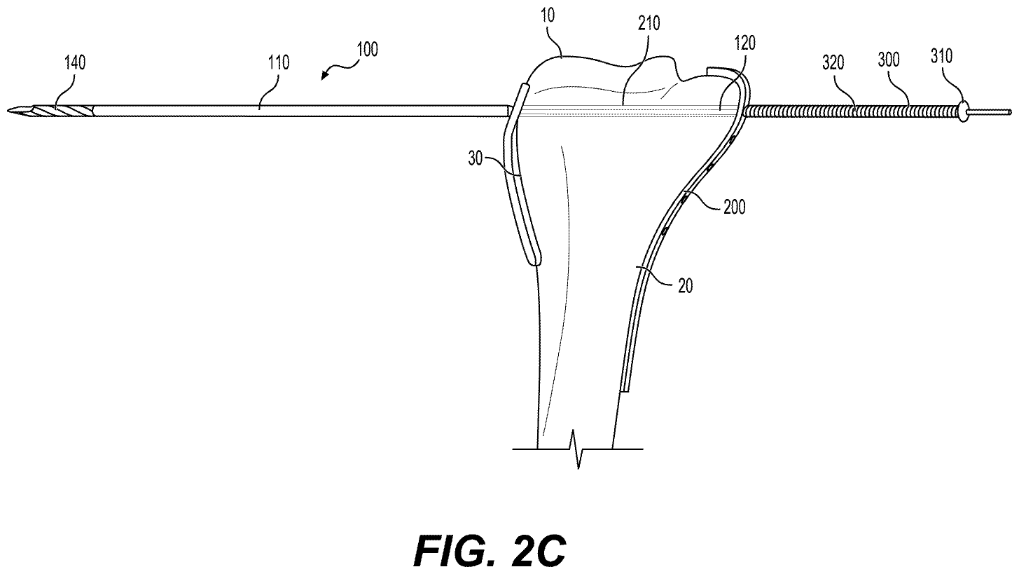

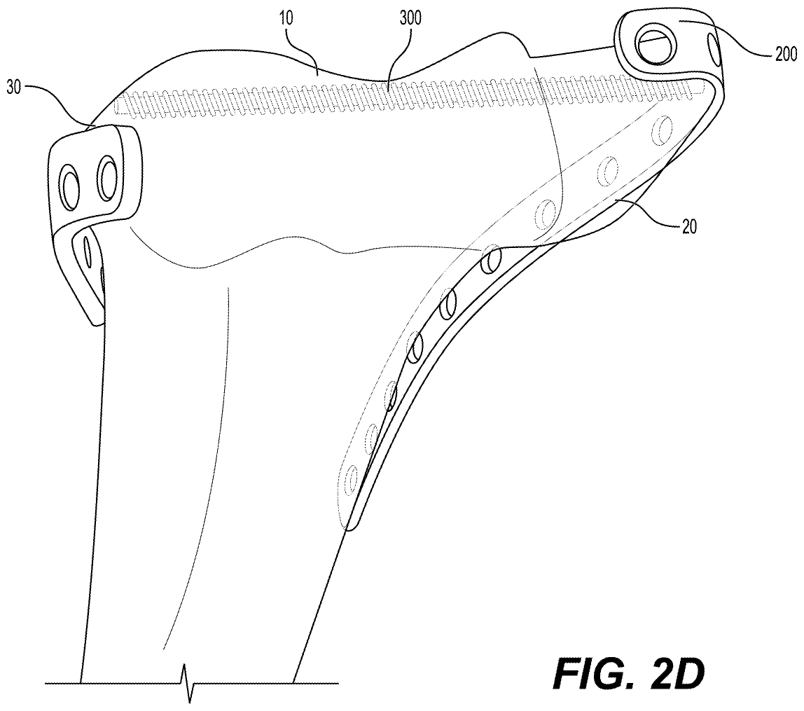

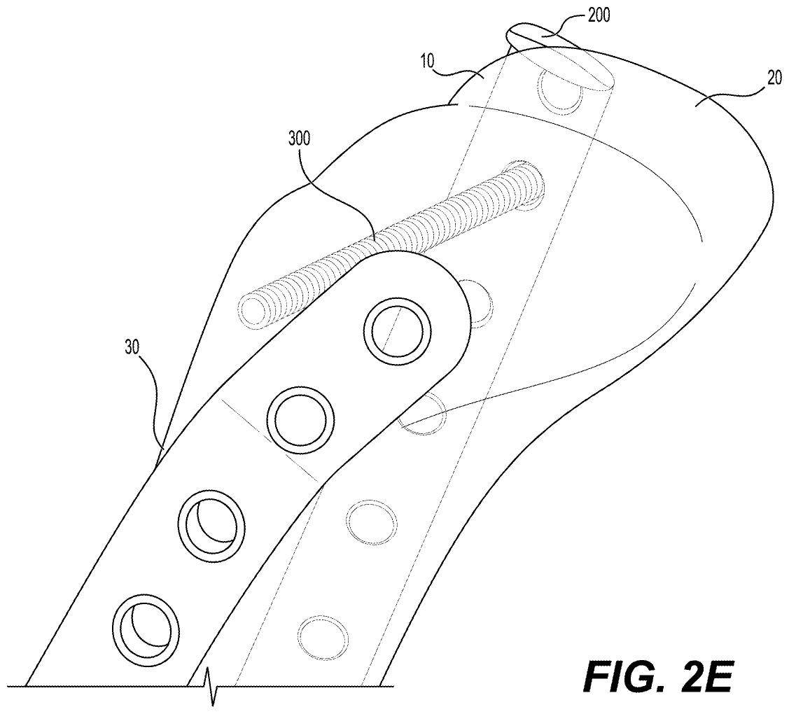

Referring to FIGS. 2A to 2D, the method may include drilling the drilling guidewire 100 through the bone 10 or, for example, drilling the drilling guidewire 100 through a hole in a bone plate 200 and through the bone 10. The drill unit 140 of the drilling guidewire 100 may contact the surface of the bone 10. A driver (such as a powered driver) may be used to drive the drill unit 140 into the bone 10, creating a channel through the bone from a first side of the bone 20 to a second side of the bone 30. The second side of the bone 30 may be different from the first side of the bone 20 where the drill unit 140 entered the bone 10.

The method may further include, after the drill unit 140 exits the second side of the bone 30, continuing to move the remainder of the first portion 110 of the drilling guidewire 100 through the channel 210 in the bone 10 until only the second portion 120 of the drilling guidewire 100 is disposed within the bone 10, as shown in FIG. 2B. Additionally, the second portion 120 may extend through the channel 210 in the bone 10 and through a hole in the bone plate 200. The second portion 120 may remain in the channel 210 in the bone 10 and through the hole in the bone plate 200 to provide guidance for implanting a bone fastener 300, for example, a cannulated bone fastener 300.

The method may additionally include sliding a bone fastener 300 over the second portion 120 of the drilling guidewire 100 as shown in FIG. 2C. Although FIG. 2C shows the bone fastener 300 sliding onto the second portion 120 of the drilling guidewire 100 after the first portion 110 has exited the channel 210, the bone fastener 300 may slide onto the second portion 120 of the drilling guidewire 100 while the first portion 110 is still in the channel 210 or before the first portion 120 contacts the surface of the bone 10.

In some examples, the bone fastener 300 may include a cannulation along its longitudinal axis and may be configured to receive the second portion 120 of the drilling guidewire 100. The bone fastener 300 may be driven into the channel 210 in the bone 10 using a power tool or a handheld device.

In some examples, the bone fastener 300 may include threads. The bone fastener 300 may have a major diameter corresponding to the diameter of the bone fastener 300 including the diameter of the threads. The bone fastener 300 may also have a minor diameter corresponding to the diameter of the bone fastener 300 not including the diameter of the threads. The minor diameter of the bone fastener 300 may be substantially the same as the diameter of the first portion 110 of the drilling guidewire 100. This may result in the minor diameter of the bone fastener 300 being substantially the same as the diameter of the channel 210 drilled through the bone 10.

In some examples, the major diameter of the bone fastener 300 may be greater than the diameter of the first portion 110 and the diameter of the channel 210 through the bone 10. This may allow the threads of the bone fastener 300 to engage with the bone 10, causing the bone fastener 300 to be secured in the channel 210.

As can be seen in FIGS. 2D and 2E, the bone fastener 300 may traverse the channel 210 in the bone 10 from one side of the bone to the other to secure the bone plate 200 in place. The drilling guidewire 100 may then be removed from the bone fastener 300.

In some examples, the method may further include positioning a second bone plate on a second side of the bone. The second bone plate may include one or more holes for receiving a bone fastener. In some examples, the method may include drilling a channel 210 with the drilling guidewire 100 through a hole in a first bone plate 200, through the bone 10, but not through the second bone plate, as shown in FIGS. 2D and 2E. In this case, in some examples, the methods of securing the first bone plate 200 as previously discussed may be used to secure the second bone plate to the bone. In other examples, the method may include drilling a channel 210 with the drilling guidewire 100 through a hole in a first bone plate 200, through the bone 10, and out the hole in the second bone plate.

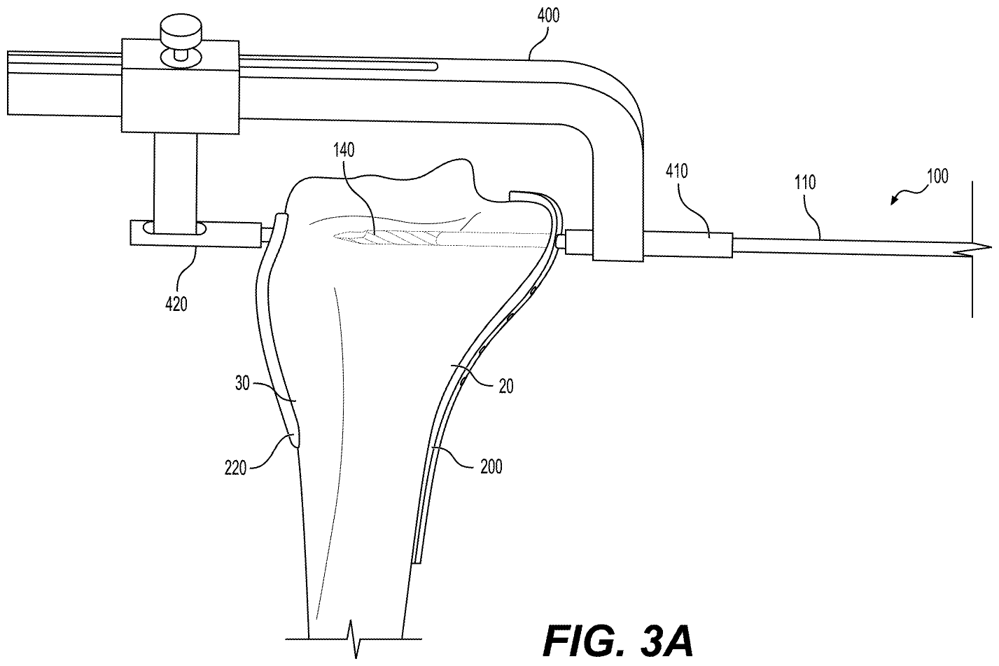

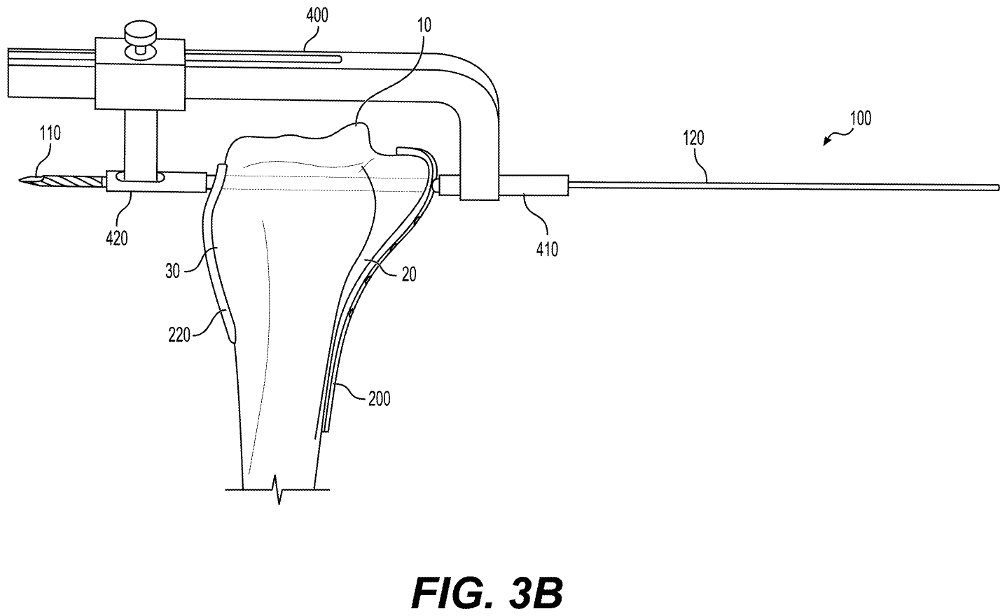

Referring to FIGS. 3A and 3B, in some examples, a clamp 400 may be used for better accuracy and ensuring the drilling guidewire 100 will not miss the hole in the second bone plate 220. The clamp 400 may be positioned around the bone 10 (e.g., from the first side of the bone 20 to the second side of the bone 30) as shown in FIG. 3A. The clamp 400 may include one or more guide channels for guiding the drilling guidewire 100. For example, the clamp may include a first guide channel 410 and a second guide channel 420.

The method may include positioning the first guide channel 410 in line with a hole in the first bone plate 200. Additionally, the method may include positioning the second guide channel 420 in line with a hole in the second bone plate 220. The clamp 400 may be adjustable, so that the distance between the first guide channel 410 and the second guide channel 420 may be modified depending on the distance from the first side of the bone 20 to the second side of the bone 30.

Referring to FIG. 3A, the drilling guidewire 100 may be positioned in the first guide channel 410 of the clamp 400. The first portion 110 of the drilling guidewire 100 may then be drilled through the bone 10, for example, using a driver, to create a channel 210 through the bone. The first portion 110 of the drilling guidewire 100 may then exit a second side of the bone 30, through a hole in the second bone plate 220, and through the second guide channel 420 of the clamp 400 as shown in FIG. 3B. The clamp 400 may be removed so that the bone fastener 300 may be slid over the second portion 120 of the drilling guidewire 100.

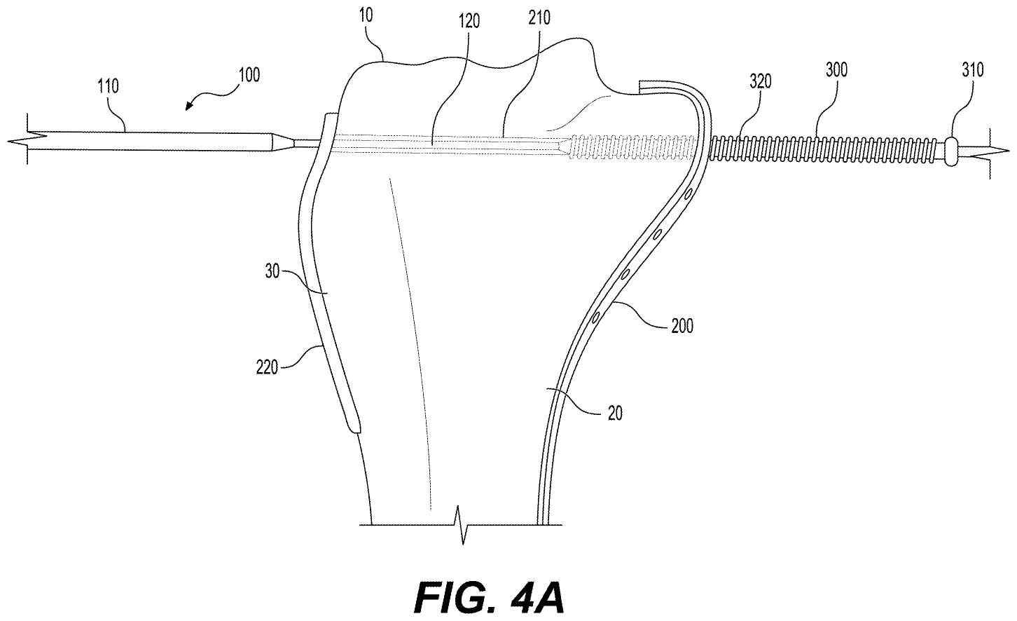

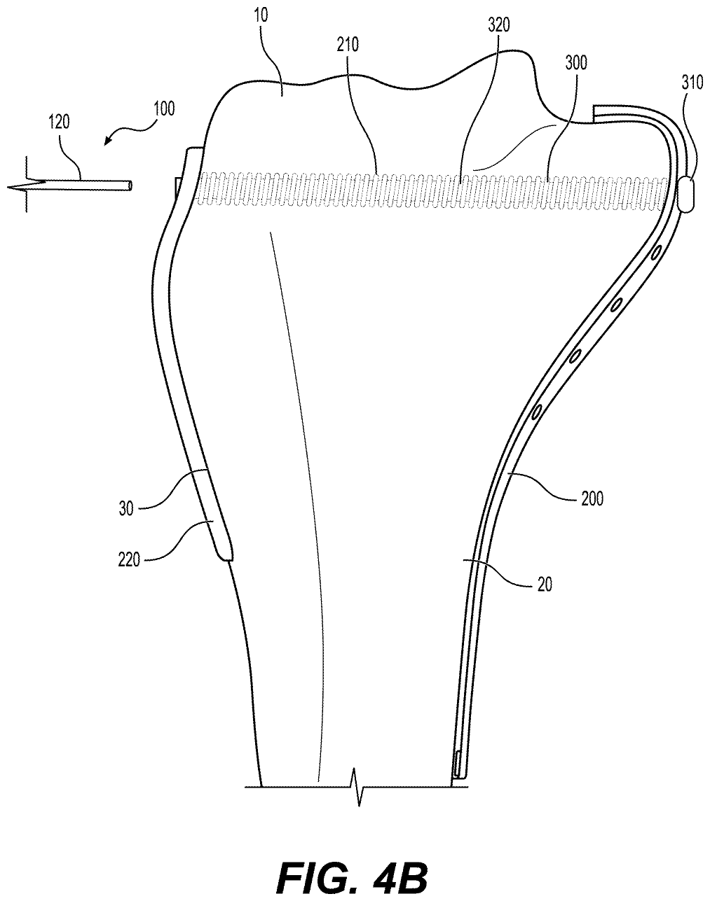

The drilling guidewire 100 may be moved through the bone so only the second portion 120 of the guidewire 100 is still in the channel 210 in the bone 10. The bone fastener 300 may then slide over the second portion 120 of the drilling guidewire 100 and may be driven through the hole in the first bone plate 200, through the channel 210 in the bone 10, and through the hole in the second bone plate 220 as shown in FIGS. 4A and 4B. The drilling guidewire 100 may then be removed from the cannulation in the bone fastener 300.

In some examples, the first or second bone plate 200, 220 and the bone fastener 300 may be locking. For example, the hole in the first or second bone plate 200, 220 may include threads configured to mate with threads on at least a portion of the bone fastener 300, in essence locking the bone fastener 300 to the first or second bone plate 200, 220.

It can be understood that many types of bone fasteners (e.g., a nail, a screw) can be used in the methods disclosed herein. For example, as shown in FIGS. 2C-2E, 4A, and 4B, the bone fastener 300 may include a head 310 and a shaft 320, where the diameter of the head 310 may be larger than the diameter of the shaft 320. In some examples, the head 310 of the bone fastener 300 may be larger than a hole in the first or second bone plate 200, 220. The larger diameter of the head 310 allows the bone fastener 300 to secure the first or second bone plate 200, 220 to the bone. In some examples, the head 310 of the bone fastener 300 may be substantially the same size as the hole in the first or second bone plate 200, 220. The head 310 of the bone fastener 300 may include threads configured to mate with threads in the hole of the first or second bone plate 200, 220.

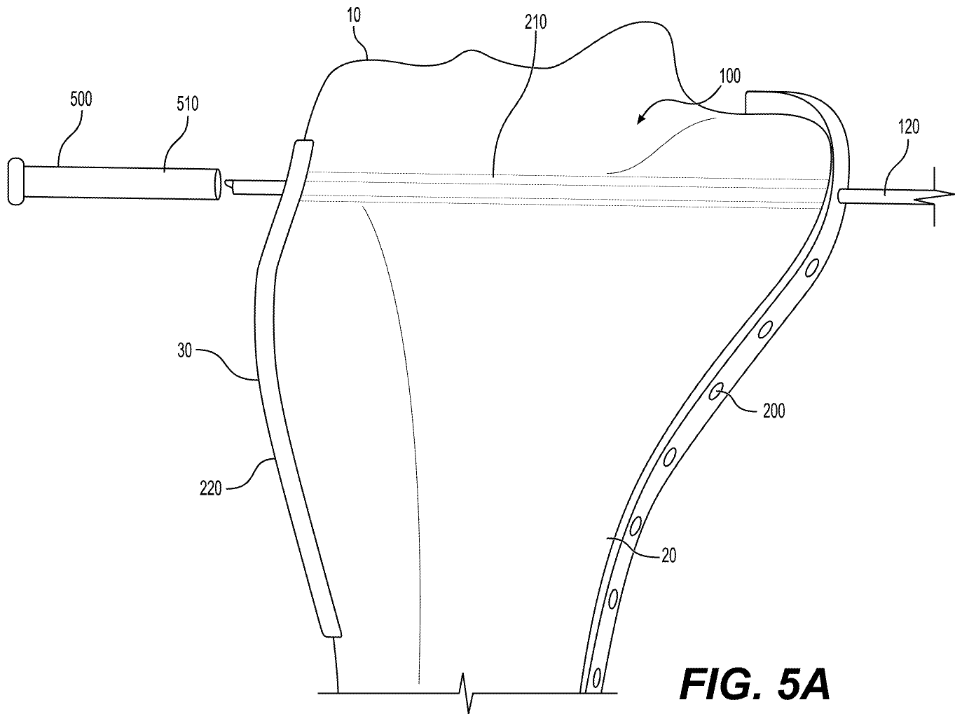

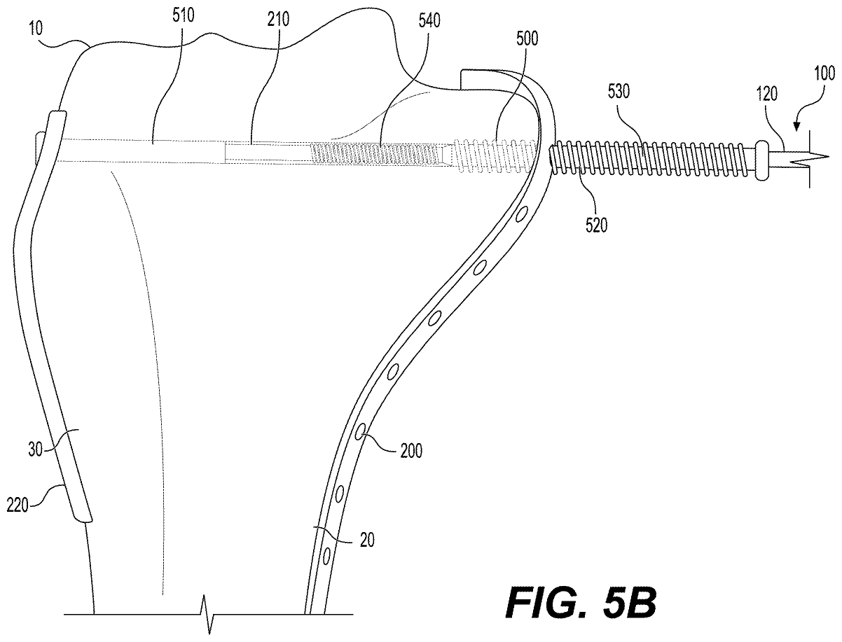

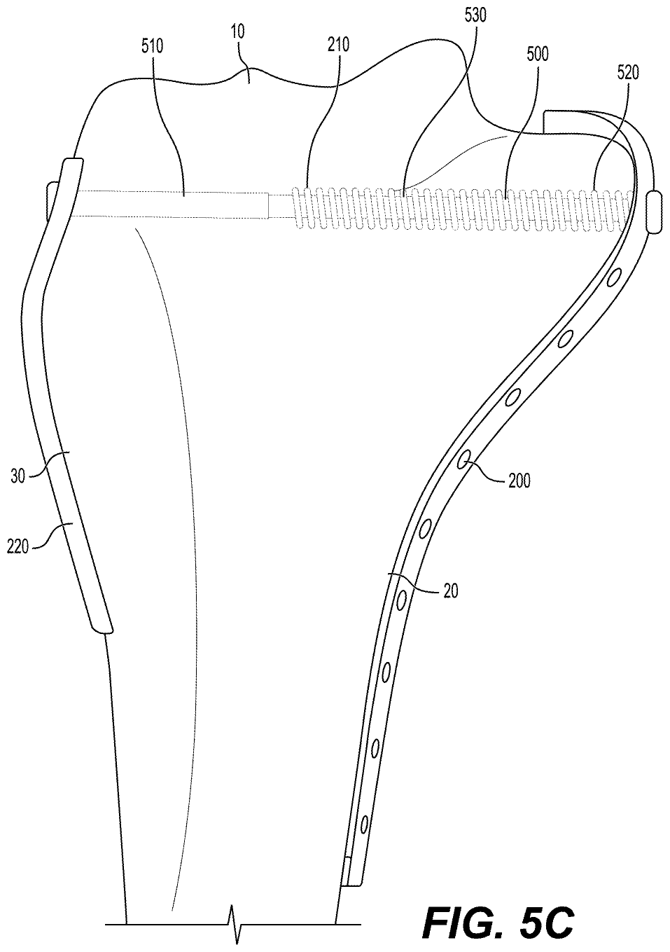

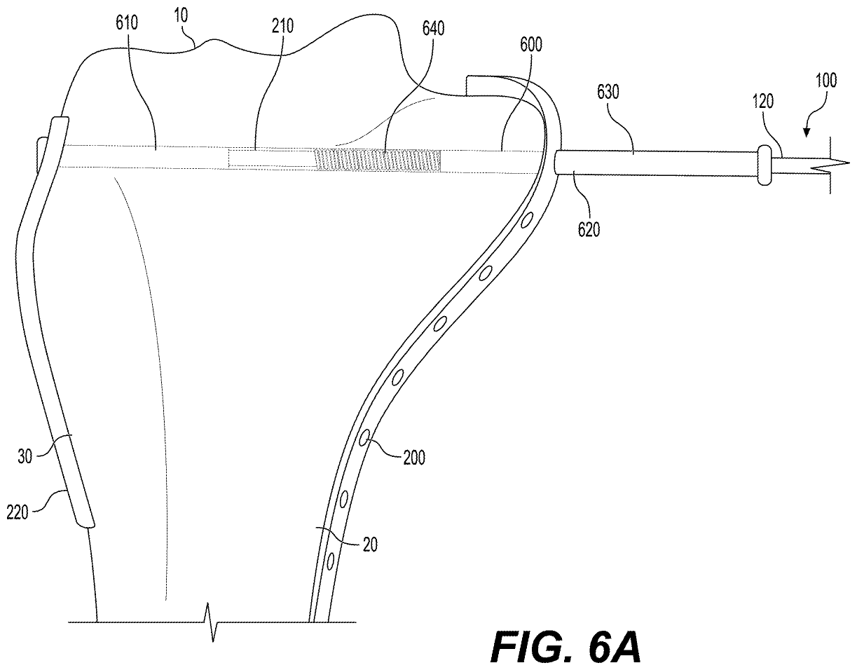

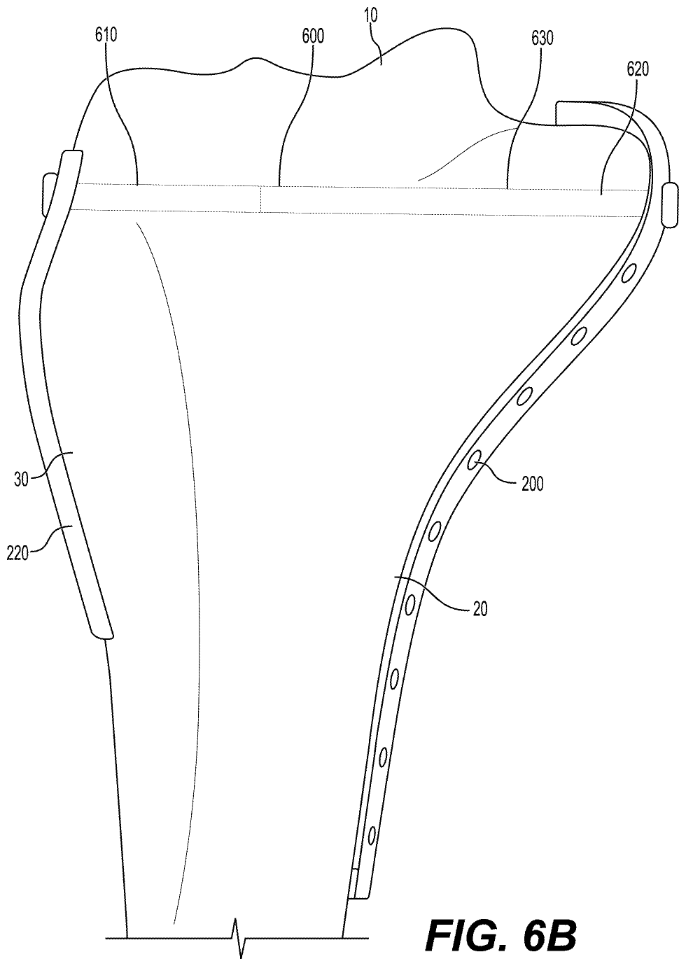

Referring to FIGS. 5A, 5B, 5C, 6A, and 6B, the bone fastener 500 may include a sleeve portion 510 and a screw portion 520. The sleeve portion 510 and the screw portion 520 may be configured to mate with each other. The screw portion 520 may include a first portion 530 and a second portion 540 where the diameter of the first portion 530 may be greater than the diameter of the second portion 540. In some examples, the diameter of the first portion 530 may be substantially the same as the outer diameter of the sleeve portion 510 so that when the sleeve portion 510 is mated with the screw portion 520, the bone fastener 500 has one generally uniform outer diameter along its length.

In some examples, as shown in FIGS. 5B and 5C, the first portion 530 and the second portion 540 of the screw portion 520 may contain threads. In other examples, as shown in FIGS. 6A and 6B, the second portion 640 of the screw portion 620 may contain threads and the first portion 630 of the screw portion 620 may not contain threads. For example, the first portion 630 may contain a smooth outer surface so that the screw portion 620 can easily glide into the channel 210 in the bone 10.

The sleeve portion 510, 610 may include a cannulation configured to receive the second portion 540, 640 of the screw portion 520, 620. In some examples, the diameter of the cannulation of the sleeve portion 510, 610 may be substantially the same as the diameter of the second portion 540, 640 of the screw portion 520, 620. The cannulation may also be configured to receive the second portion 120 of the drilling guidewire 100. For example, the cannulation of the sleeve portion 510, 610 may include internal-threads configured to mate with threads of the second portion 540, 640 of the screw portion 520, 620. However, other mating mechanisms may be used. In some examples, the outer diameter of the sleeve portion 510, 610 may be threaded. In some examples, the outer diameter of the sleeve portion 510, 610 may not be threaded, but it may have a smooth outer surface as shown in the figures.

Referring back to FIGS. 5A, 5B, 5C, 6A, and 6B, once the first portion 110 of the drilling guidewire 100 has exited the second side of the bone 30, the method may further include severing the first portion 110 of the drilling guidewire 100 from the second portion 120 of the drilling guidewire 100. For example, in order to insert a sleeve portion 510, 610 into the channel 210 from the second side of the bone 30 and a screw portion 520, 620 into the channel 210 from the first side of the bone 20, or to insert a screw portion 520, 620 into the channel 210 from the second side of the bone 30 and a sleeve portion 510, 610 into the channel 210 from the first side of the bone 20, the first portion 110 of the drilling guidewire 100 may need to be removed so that the sleeve portion 510, 610 or screw portion 520, 620 can slide onto the drilling guidewire 100 and into the channel 210 in the bone 10.

The severing of the first portion 110 from the second portion 120 may include cutting the first portion 110 off of the second portion 120. In some examples, the first portion 110 of the drilling guidewire 100 and the second portion 120 of the drilling guidewire 100 may be removably coupled to one another. Severing the drilling guidewire 100 may include removing/detaching the first portion 110 of the drilling guidewire 100 from the second portion 120 of the drilling guidewire 100.

Once the first portion 110 of the drilling guidewire 100 is severed from the second portion 120, the sleeve portion 510, 610 of the bone fastener 500, 600 may slide over the first portion 110 of the drilling guidewire 100 on the severed side. The sleeve portion 510, 610 may then be positioned in a hole in the second bone plate 220 and driven into the channel 210 in the bone 10 from the second side of the bone 30. The method may further include inserting the screw portion 520, 620 over the first portion 110 of the drilling guidewire 100 and driving the screw portion 520, 620 into the first side of the bone 20. The screw portion 520, 620 may meet the sleeve portion 510, 610 and the two may couple together as shown in FIGS. 5C and 6B. In some examples, the method may include screwing the screw portion 520, 620 into the sleeve portion 510, 610. In other examples, any other suitable coupling mechanisms can be used.

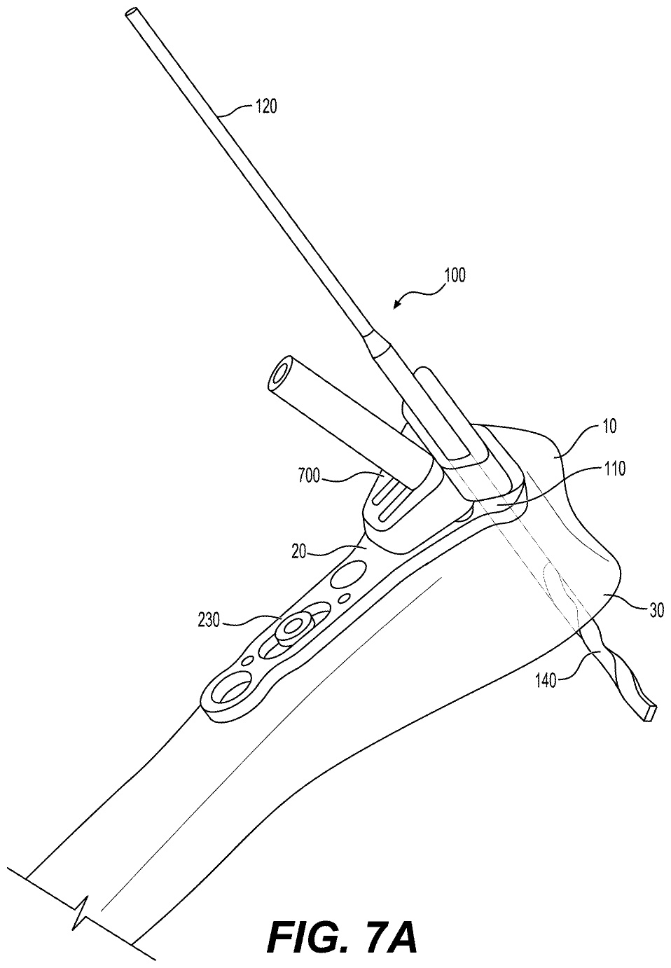

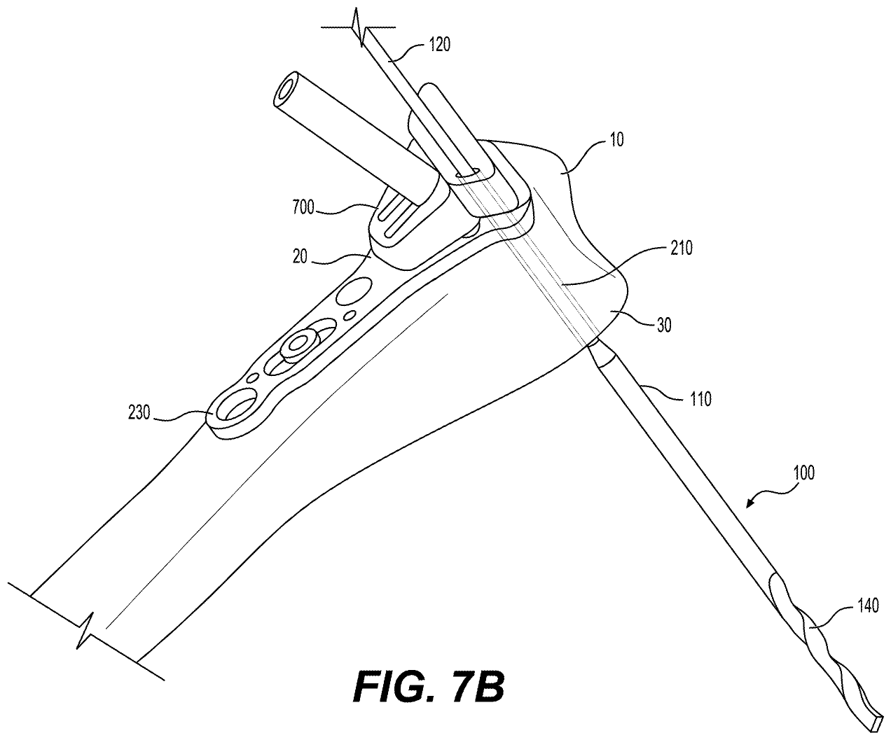

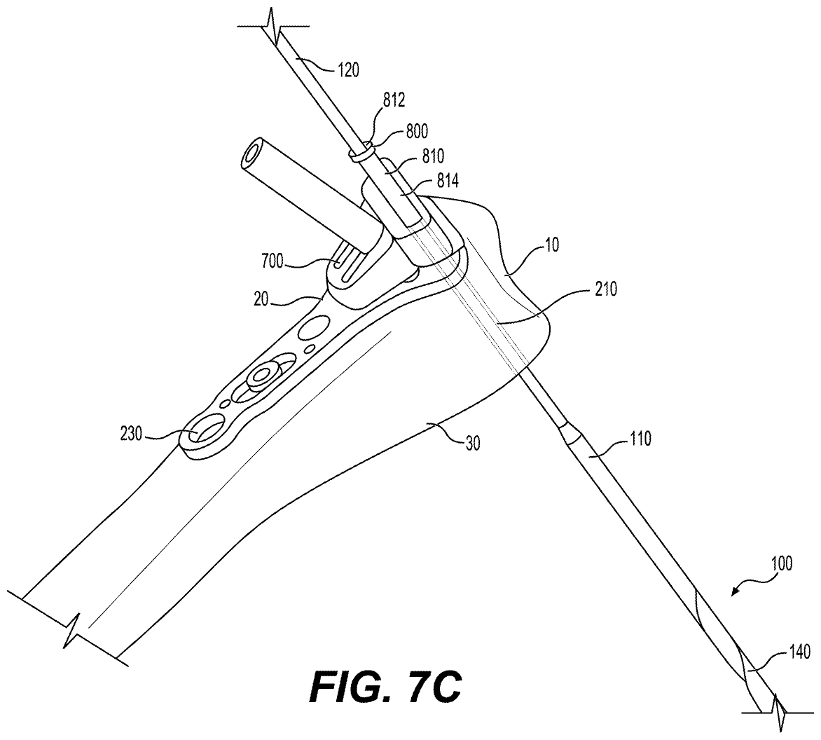

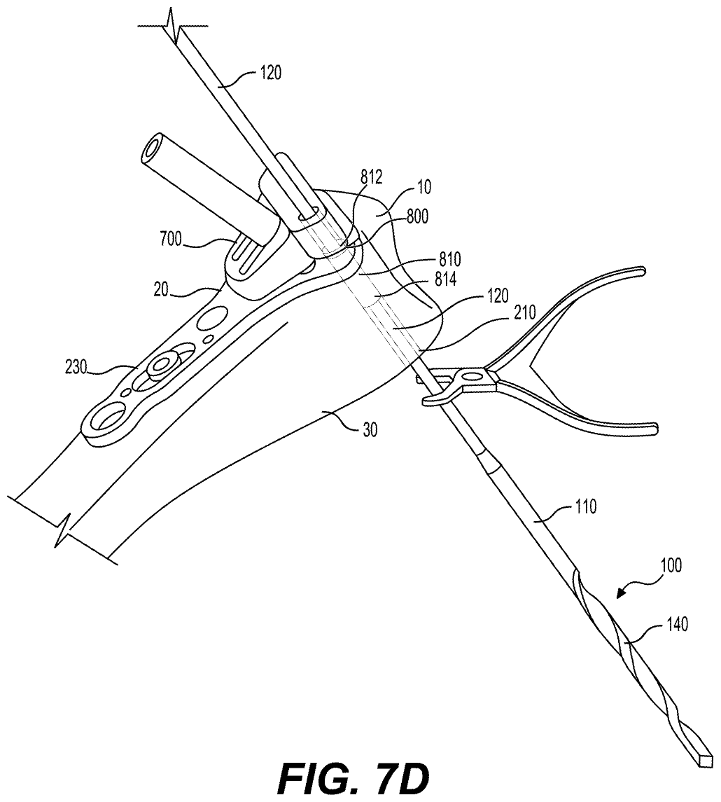

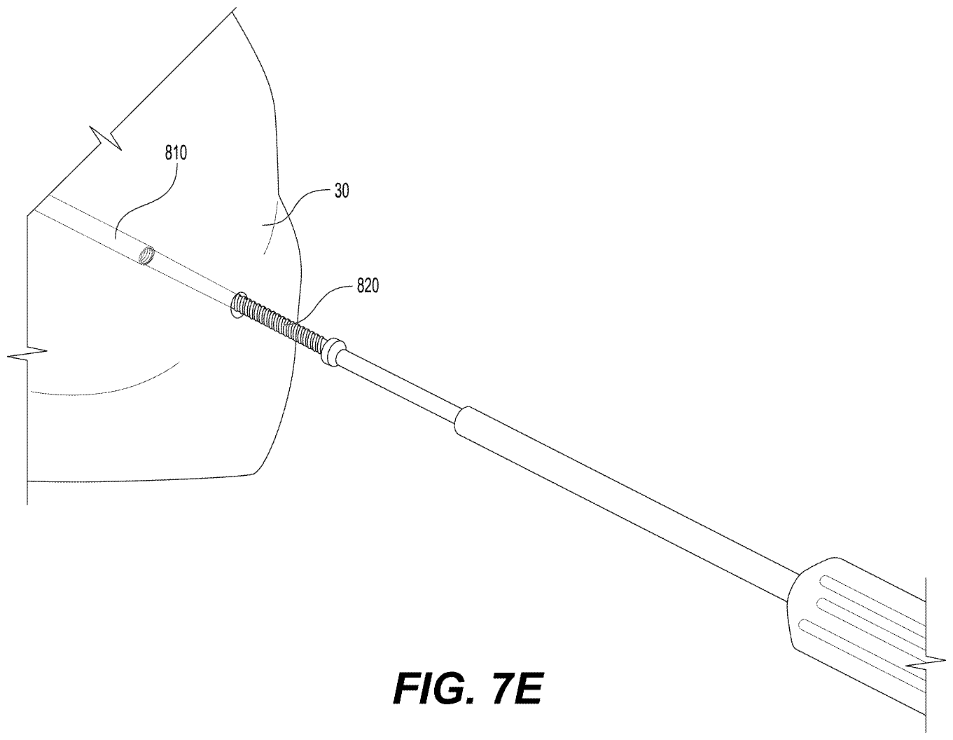

In some examples, a method of inserting a compression device 800 into a bone 10 is provided herein as displayed in FIGS. 7A through 7E. The method may include positioning a bone plate 230 on a first side of a bone 20, for example, the distal radius. The bone plate 230 may include one or more holes configured to receive a compression device 800. The bone plate 230 may additionally include one or more holes configured to receive a bone fastener.

The compression device 800 may include a sleeve portion 810 and a screw portion 820. In some examples, both the sleeve portion 810 and the screw portion 820 may include cannulations. The screw portion 820 and the sleeve portion 810 may be configured to mate with each other. For example, the cannulation of the sleeve portion 810 may be configured to receive the outer diameter of the screw portion 820.

In some examples, the sleeve portion 810 may include a head 812 and a shaft 814. In some examples, the head 812 may include threads configured to mate with threads in the hole of the bone plate 230. In some example, the diameter of the head 812 may be greater than the diameter of the shaft 814.

Referring to FIG. 7A, the method may include drilling a channel 210 through a hole in the bone plate 230 and through the bone 10 using the drilling guidewire 100 as disclosed herein. In some examples, a targeting guide 700 may be used to position the drilling guidewire 100 before drilling into the bone 10 to ensure proper alignment with the bone plate 230. The drill unit 140 of the first portion 110 of the drilling guidewire 100 may contact the bone 10 and through the use of a driver create a channel 210 through the bone 10. Once the drill unit 140 exits the second side of the bone 30, the first portion 110 of the drilling guidewire 100 may continue to exit the channel 210 in the bone until only the second portion 120 of the drilling guidewire 100 is disposed in the bone 10 as shown in FIG. 7B.

Referring to FIG. 7C, the sleeve portion 810 of the compression device 800 may slide over the second portion 120 of the drilling guidewire 100. The sleeve portion 810 then may be driven into the bone 10. In some examples, the head 812 of the sleeve portion 810 may be locked in to the hole in the bone plate 230.

The method may further include severing the first portion 110 of the drilling guidewire 100 from the second portion 120 as shown in FIG. 7D. As discussed previously, it can be understood that other severing methods may be used alternatively or in addition to what is shown in FIG. 7D. This allows the cannulated screw portion 820 to fit over the second portion 120 of the drilling guidewire 100 while the second portion 120 is still disposed in the bone 10.

After severing the first portion 110 from the second portion 120, the screw portion 820 of the compression device 800 may slide over the severed side of the second portion 120 of the drilling guidewire 100 as shown in FIG. 7E. The screw portion 820 may then be driven into the channel 210 in the bone 10 from the second side of the bone 30. The method may further include inserting the screw portion 820 into the sleeve portion 810 and securing the two together. In some examples, the method may include tightening the screw portion 820 into the sleeve portion 810 until a fracture is reduced. For example, by tightening the screw portion 820 with the sleeve portion 810, the compression device 800 may compress the bone 10 joining the fracture points together.

Although it has been described above with respect to FIGS. 7A to 7D that the sleeve portion 810 is inserted from the first side of the bone 20, and the screw portion 820 is inserted from the second side of the bone 30 after severing the first portion 110 of the drilling guidewire 100 from the second portion 120, in some other examples, the screw portion 820 can be inserted from the first side of the bone 20 and the sleeve portion 810 can be inserted from the second side of the bone 30 after severing the first portion 110 of the drilling guidewire 100 from the second portion 120.

For example, once the first portion 110 of the drilling guidewire 100 exits the channel 210 in the bone 10 until only the second portion 120 of the drilling guidewire 100 is disposed in the bone 10, the screw portion 820 of the compression device 800 may slide over the second portion 120 of the drilling guidewire 100. The sleeve portion 810 then may be driven into the bone 10. In some examples, the head of the screw portion 820 may be locked in to the hole in the bone plate 230.

The method may further include after severing the first portion 110 from the second portion 120, the sleeve portion 810 of the compression device 800 may slide over the severed side of the second portion 120 of the drilling guidewire 100. The sleeve portion 810 may then be driven into the channel 210 in the bone 10 from the second side of the bone 30. The method may further include screwing the screw portion 820 into the sleeve portion 810 and securing the two together.

Other configurations/features/characteristics of the method may be similar to and/or the same as the ones described above with respect to the methods of inserting a compression device into a bone that are described with respect to FIGS. 7A through 7E (e.g., other steps, structure of the sleeve/screw portion), and, thus, duplicate description may be omitted.

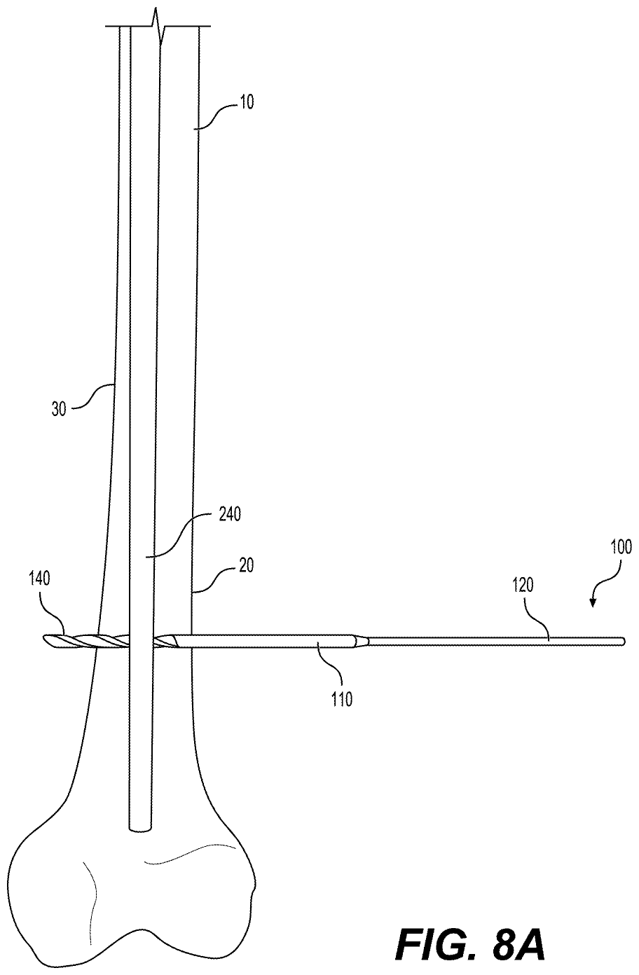

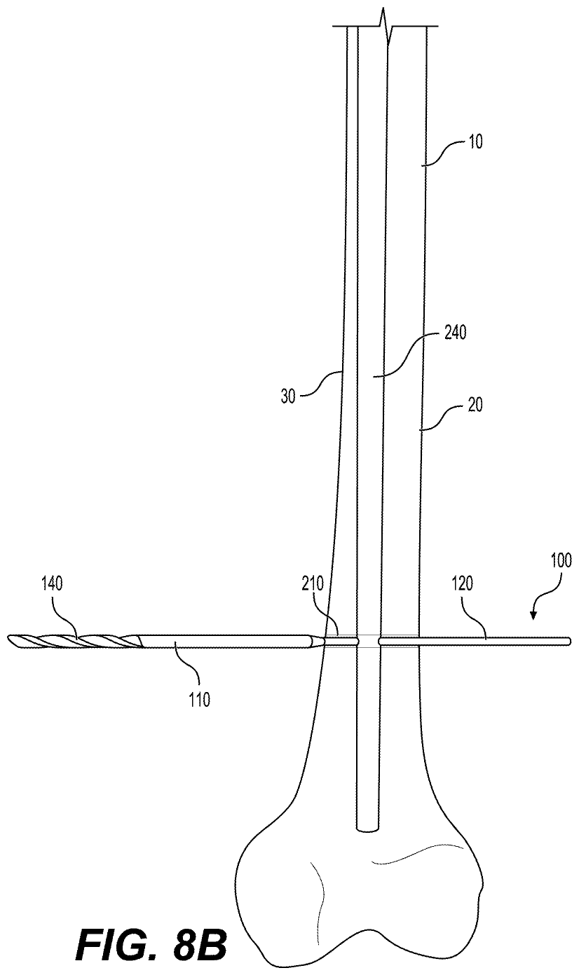

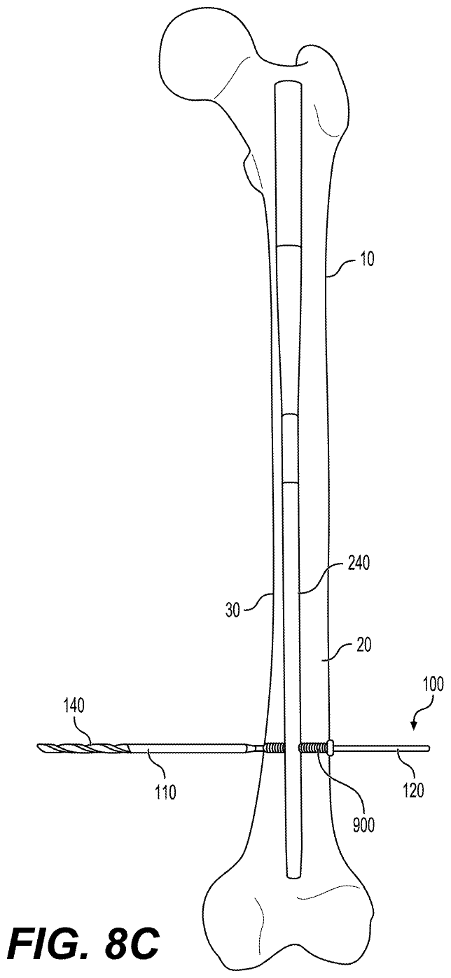

In some examples, a method of securing an intramedullary device 240 using a drilling guidewire 100 of the present disclosure is provided. Exemplary steps of the method are shown in FIGS. 8A through 8C. The method may include inserting an intramedullary device 240, such as an intramedullary nail or rod, into the medullary cavity of a bone 10. Some intramedullary devices require the insertion of screws to stabilize the device. As shown in FIG. 8A, the method may include drilling a channel through the bone 10 and through a hole in the intramedullary device 240 using the drilling guidewire 100 disclosed herein. The drilling guidewire 100 may be moved through the bone 10 until only the second portion 120 of the drilling guidewire 100 is disposed in the bone 10 and the hole in the intramedullary device 240 as show in FIG. 8B. In some examples, a bone fastener 900 slides over the second portion 120 of the drilling guidewire 100. The bone fastener 900 may be driven into the channel 210 in the bone 10 and through the hole in the intramedullary device 240 as show in FIG. 8C. The method may then include removing the drilling guidewire 100 from the bone 10.

Other configurations/features/characteristics/steps of the method of securing an intramedullary device may be similar to and/or the same as the ones described above with respect to the method of inserting a bone fastener through a bone (e.g., types of bone fasteners, the drilling guidewire, drilling methods) and, thus, duplicate description may be omitted.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

### Embodiments

Various aspects of the subject matter described herein are set out in the following numbered embodiments:

Embodiment 1. A method of inserting a bone fastener through a bone, the method comprising: positioning a first bone plate having a first hole on a first side of a bone; drilling a channel through the bone from the first side of the bone to a second side of the bone using a drilling guidewire, wherein the drilling guidewire comprises a first portion having a drill unit and a second portion, the first portion comprising a first end and a second end, the second portion extending from the second end of the first portion, and wherein a diameter of the first portion is greater than a diameter of the second portion, wherein the first end of the first portion is positioned in the first hole of the first bone plate prior to drilling the channel through the bone so that the drilling guidewire is fed through the first hole in the first bone plate; placing at least a part of the second portion of the drilling guidewire within the bone; sliding a bone fastener over the second portion of the drilling guidewire, wherein the bone fastener comprises a cannulation configured to receive the second portion of the drilling guidewire; and driving the bone fastener through the first hole of the first bone plate and the channel in the bone.

Embodiment 2. The method of embodiment 1, wherein a minor diameter of the bone fastener is substantially equal to a diameter of the first portion of the drilling guidewire.

Embodiment 3. The method of any one of embodiments 1-2, wherein the bone fastener comprises a bone screw.

Embodiment 4. The method of any one of embodiments 1-3, further comprising positioning a second bone plate with a second hole on the second side of the bone.

Embodiment 5. The method of any of embodiments 1-4, wherein at least a part of the first portion of the drilling guidewire passes through the channel of the bone and through the second hole of the second bone plate.

Embodiment 6. The method of any embodiments 1-5, further comprising: wherein at least a part of the first portion of the drilling guidewire passes through the channel of the bone and through a portion of the second side of the bone that is not covered by the second bone plate.

Embodiment 7. The method of any one of embodiments 1-6, further comprising: positioning a clamp around the bone, wherein the clamp comprises a first guide channel and a second guide channel, wherein positioning the clamp around the bone comprises: positioning the first guide channel in line with the first hole in the first bone plate disposed on the first side of the bone; and positioning the second guide channel in line with the second hole in the second bone plate disposed on the second side of the bone.

Embodiment 8. The method of any one of embodiments 1-7, wherein at least a part of the first portion of the drilling guidewire passes through the first guide channel of the clamp, the first hole in the first bone plate, the channel of the bone, the second hole in the second bone plate, and the second guide channel of the clamp.

Embodiment 9. The method of any one of embodiments 1-8, further comprising: removing the clamp; severing and removing the first portion of the drilling guidewire; sliding a sleeve over the second portion of the drilling guidewire from the second side of the bone; driving the sleeve through the second hole in the second bone plate and through the channel in the bone from the second side of the bone, wherein the sleeve comprises a threaded cannulation; and securing a first portion of the bone fastener with the sleeve.

Embodiment 10. The method of any one of embodiments 1-9, wherein the bone fastener comprises: the first portion having a first diameter; and a second portion having a second diameter greater than the first diameter.

Embodiment 11. The method of embodiment 10, wherein the first diameter of the first portion of the bone fastener is substantially equal to a diameter of the threaded cannulation of the sleeve.

Embodiment 12. The method of embodiments 10 and 11, wherein the first portion of the bone fastener is threaded and the second portion of the bone fastener is non-threaded.

Embodiment 13. The method of any embodiments 1-12, further comprising: removing the clamp; and driving the bone fastener through the first hole in the first plate, the channel in the bone, and the second hole in the second bone plate.

Embodiment 14. The method of any one of embodiments 1-13, wherein at least a portion of the bone fastener comprises an external thread and wherein at least one of the first hole of the first bone plate and the second hole of the second bone plate comprises an internal thread configured to mate with the external thread of the bone fastener.

Embodiment 15. The method of any of embodiments 1-14, wherein the bone fastener comprises a shaft and a head, wherein a diameter of the head is greater than a diameter of the shaft.

Embodiment 16. The method of embodiment 15, wherein the diameter of the head is greater than the diameter of the first hole in the first bone plate so the head of the bone fastener secures the first bone plate to the bone.

Embodiment 17. The method of any of embodiments 1-16, wherein the bone comprises a humerus, a radius, an ulna, a clavicle, a tibia, a fibula, a metacarpal, a metatarsal, a phalange, or a femur.

Embodiment 18. The method of any one of embodiments 1-17, wherein the diameter of the first portion of the drilling guidewire is in a range of about 1.2 mm to about 6.5 mm.

Embodiment 19. The method of any one of embodiments 1-18, wherein the diameter of the second portion of the drilling guidewire is in a range of about 0.5 mm to about 4.5 mm.

Embodiment 20. The method of any one of embodiments 1-19, wherein the diameter of the first portion of the drilling guidewire is greater than a diameter of the cannulation of the bone fastener.

Embodiment 21. A method of securing an intramedullary device, the method comprising drilling a channel through a bone from a first side of the bone to a second side of the bone using a drilling guidewire, wherein the intramedullary device is disposed within bone between the first side of the bone and the second side of the bone and wherein the channel is disposed through a hole in the intramedullary device, wherein the drilling guidewire comprises a first portion having a drill unit and a second portion, the first portion comprising a first end and a second end, the second portion extending from the second end of the first portion, and wherein a diameter of the first portion is greater than a diameter of the second portion; placing at least a part of the second portion of the drilling guidewire within the bone; sliding a bone fastener over the second portion of the drilling guidewire, wherein the bone fastener comprises a cannulation configured to receive the second portion of the drilling guidewire; and driving the bone fastener through the channel in the bone and through the hole in the intramedullary device.

Embodiment 22. The method of embodiment 21, wherein the drill unit comprises two or more flutes.

Embodiment 23. A method of inserting a compression device in a bone, wherein the compression device comprises a screw and a sleeve, the method comprising: positioning a first bone plate having a first hole on a first side of a bone; drilling a channel through the bone from the first side of the bone to a second side of the bone using a drilling guidewire, wherein the drilling guidewire comprises a first portion having a drill unit and a second portion, the first portion comprising a first end and a second end, the second portion extending from the second end of the first portion, and wherein a diameter of the first portion is greater than a diameter of the second portion, wherein the first end of the first portion is positioned in the first hole of the first bone plate prior to drilling the channel through the bone so that the drilling guidewire is fed through the first hole in the first bone plate; placing at least a part of the second portion of the drilling guidewire within the bone; sliding the sleeve over the second portion of the drilling guidewire, wherein the sleeve comprises a cannulation configured to receive the second portion of the drilling guidewire and wherein the cannulation is configured to receive the screw; driving the sleeve through the first hole of the first bone plate and the channel in the bone; severing and removing the first portion of the drilling guidewire; sliding the screw over the second portion of the drilling guidewire from the second side of the bone, wherein the screw comprises a cannulation configured to receive the second portion of the drilling guidewire; screwing the screw into the channel in the bone; and securing the screw with the sleeve.

Embodiment 24. The method of embodiment 23, wherein securing the screw with the sleeve comprises tightening the screw into the sleeve until a fracture in the bone is reduced.

As used herein, “about,” “approximately” and “substantially” are understood to refer to numbers in a range of numerals, for example the range of −10% to +10% of the referenced number, preferably −5% to +5% of the referenced number, more preferably-1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

Additionally, in describing the components of the system of the present disclosure, there may be terms used like first, second, third, and fourth. These terms may be used for the purpose of differentiating one component from the other, but not to imply or suggest the substances, order, sequence, or number of the components.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of inserting a bone fastener through a bone, the method comprising:
positioning a first bone plate having a first hole on a first side of a bone;
drilling a channel through the bone from the first side of the bone to a second side of the bone using a drilling guidewire, wherein the drilling guidewire comprises a first portion having a drill unit and a second portion, the first portion comprising a first end and a second end, the second portion extending from the second end of the first portion, and wherein a diameter of the first portion is greater than a diameter of the second portion, wherein the first end of the first portion is positioned in the first hole of the first bone plate prior to drilling the channel through the bone so that the drilling guidewire is fed through the first hole in the first bone plate;
placing at least a part of the second portion of the drilling guidewire within the bone;
sliding a bone fastener over the second portion of the drilling guidewire, wherein the bone fastener comprises a cannulation configured to receive the second portion of the drilling guidewire; and
driving the bone fastener through the first hole of the first bone plate and the channel in the bone.

2. The method of claim 1, wherein a minor diameter of the bone fastener is substantially equal to a diameter of the first portion of the drilling guidewire.

3. The method of claim 1, wherein the bone fastener comprises a bone screw.

4. The method of claim 1, comprising positioning a second bone plate with a second hole on the second side of the bone.

5. The method of claim 4, wherein at least a part of the first portion of the drilling guidewire passes through the channel of the bone and through the second hole of the second bone plate.

6. The method of claim 4, wherein at least a part of the first portion of the drilling guidewire passes through the channel of the bone and through a portion of the second side of the bone that is not covered by the second bone plate.

7. The method of claim 4, further comprising:
positioning a clamp around the bone, wherein the clamp comprises a first guide channel and a second guide channel, wherein positioning the clamp around the bone comprises:
positioning the first guide channel in line with the first hole in the first bone plate disposed on the first side of the bone; and
positioning the second guide channel in line with the second hole in the second bone plate disposed on the second side of the bone.

8. The method of claim 7, wherein at least a part of the first portion of the drilling guidewire passes through the first guide channel of the clamp, the first hole in the first bone plate, the channel of the bone, the second hole in the second bone plate, and the second guide channel of the clamp.

9. The method of claim 7, further comprising:
removing the clamp;
severing and removing the first portion of the drilling guidewire;
sliding a sleeve over the second portion of the drilling guidewire from the second side of the bone;
driving the sleeve through the second hole in the second bone plate and through the channel in the bone from the second side of the bone, wherein the sleeve comprises a threaded cannulation; and
securing a first portion of the bone fastener with the sleeve.

10. The method of claim 9, wherein the bone fastener comprises:
the first portion having a first diameter; and
a second portion having a second diameter greater than the first diameter.

11. The method of claim 10, wherein the first diameter of the first portion of the bone fastener is substantially equal to a diameter of the threaded cannulation of the sleeve.

12. The method of claim 10, wherein the first portion of the bone fastener is threaded and the second portion of the bone fastener is non-threaded.

13. The method of claim 7, further comprising:
removing the clamp; and
driving the bone fastener through the first hole in the first plate, the channel in the bone, and the second hole in the second bone plate.

14. The method of claim 13, wherein at least a portion of the bone fastener comprises an external thread and wherein at least one of the first hole of the first bone plate and the second hole of the second bone plate comprises an internal thread configured to mate with the external thread of the bone fastener.

15. The method of claim 1, wherein the bone fastener comprises a shaft and a head, wherein a diameter of the head is greater than a diameter of the shaft.

16. The method of claim 15, wherein the diameter of the head is greater than the diameter of the first hole in the first bone plate so the head of the bone fastener secures the first bone plate to the bone.

17. The method of claim 1, wherein the bone comprises a humerus, a radius, an ulna, a clavicle, a tibia, a fibula, a metacarpal, a metatarsal, a phalange, or a femur.

18. The method of claim 1, wherein the diameter of the first portion of the drilling guidewire is in a range of about 1.2 mm to about 6.5 mm.

19. The method of claim 1, wherein the diameter of the second portion of the drilling guidewire is in a range of about 0.5 mm to about 4.5 mm.

20. The method of claim 1, wherein the diameter of the first portion of the drilling guidewire is greater than a diameter of the cannulation of the bone fastener.
